(19) United States
(12) Patent Application Publication
(10) Pub. No.: US 2025/0262071 A1
(43) Pub. Date: Aug. 21, 2025

(54) PROSTHESIS SOCKET AND METHOD FOR PRODUCING SAME

(71) Applicant: OTTO BOCK HEALTHCARE PRODUCTS GMBH, Wien (AT)

(72) Inventors: Sonja WAGNER, Wien (AT); Alice SCHERB, Stockerau (AT); Walter LUNZER, Wien (AT)

(21) Appl. No.: 19/200,259

(22) Filed: May 6, 2025

Related U.S. Application Data
(63) Continuation of application No. 17/780,008, filed on May 25, 2022.

(30) Foreign Application Priority Data
Nov. 28, 2019 (DE) 10 2019 132 290.1

(51) Int. Cl. A61F 2/80; A61F 2/78

(52) U.S. Cl. CPC A61F 2/80; A61F 2/7812; A61F 2002/7818

(57) ABSTRACT

The invention relates to a prosthesis socket comprising a proximal entry opening and a distal end or a distal end region and a fastening system provided there for a prosthesis component, wherein the prosthesis socket has a frame consisting of a fiber-reinforced plastic material with at least one opening, a textile which spans the opening being fixed to the frame.

### CROSS REFERENCE TO RELATED APPLICATIONS

This is a continuation of U.S. patent application Ser. No. 17/780,008, filed 25 May 2022, which is a national phase application of International Application No. PCT/EP2020/083616, filed 27 Nov. 2020, which claims the benefit of German Patent Application No. 10 2019 132 290.1, filed 28 Nov. 2019, the disclosures of which are incorporated herein, in their entireties, by this reference.

### TECHNICAL FIELD

The invention relates to a prosthesis socket comprising a proximal entry opening and a distal end or a distal end region and an attachment device arranged there for a prosthesis component, the prosthesis socket comprising a frame composed of fiber-reinforced plastic having at least one free space, and to a method for production thereof.

### BACKGROUND

Prostheses replace limbs or parts of limbs that are nonexistent or no longer exist, the intention being that the prosthesis replaces the shape and/or function of the natural limb as best as possible. Various constructions and technologies are known for attachment of prostheses to the particular user. A widespread option is the fixing of prosthesis components, for example prosthesis joints and distal prosthesis components, via a prosthesis socket put on a stump. A prosthesis liner can be arranged between the prosthesis socket, which can comprise a receptacle for a prosthesis joint or some other component at its distal end, and the stump in order to reduce pressure load, to provide padding and, if applicable, to ensure a sealing effect as part of suction socket technology. Here, the prosthesis sockets are commonly in the form of custom-made, dimensionally stable components composed of fiber-reinforced plastics. The fibers are generally high-strength inorganic or organic fibers such as carbon fibers, glass fibers or aramid fibers; thermoplastics or thermosets are used as the plastic matrix. The distal end of such a prosthesis socket is commonly closed, and so the prosthesis socket extends completely around the stump. Despite the use of high-tech materials, such prosthesis sockets are comparatively heavy and allow only limited removal of moisture.

As an alternative to prosthesis sockets having closed walls, so-called frame sockets have been developed in order to reduce weight and to build pressure or reinforcement only at the necessary and appropriate points. On such frame sockets, it is for example possible to cut out an area on a back thigh, and so the socket adapts to the seating surface and the prosthesis wearer, when sitting, does not sit on the hard prosthesis socket. The frame sockets are mainly made of prepregs, which cure under vacuum and heat, or are constructed using the technique of wet lamination. Frame sockets generally have an inner socket commonly consisting of thermoplastically deformable plastics. These materials are relatively heavy and thick and thus require a large volume. Furthermore, the inner sockets are airtight, which promotes sweating. This can lead to skin irritation.

### SUMMARY

It is an object of the present invention to provide a prosthesis socket and a method for producing a prosthesis socket, by means of which it is possible to increase the wearing comfort of prosthesis sockets without reducing functionality.

According to the invention, this object is achieved by a prosthesis socket having the features of the main claim and a method having the features of the additional independent claim. Advantageous embodiments and developments of the invention are disclosed in the dependent claims, the description and the figures.

In the prosthesis socket comprising a proximal entry opening and a distal end or a distal end region and an attachment device arranged there for a prosthesis component, the prosthesis socket comprising a frame composed of a fiber-reinforced plastic having at least one free space, a textile bridging the free space is fixed to the frame. The free space is formed in the wall of the prosthesis socket, thereby saving material of the dimensionally stable frame. As a result, the weight of the prosthesis socket is reduced. To continue to enable the stump to be supported within the socket, a textile is fixed to the frame in order to at least partially cover or fill the free space and to achieve at least a reduction in the area of the free space or window.

Advantageously, the textile completely bridges the free space and covers it completely, meaning that the window is closed and what arises in this region of the prosthesis socket is a closed cross-section composed of frame, for example composed of a fiber-reinforced plastic, and a textile attached thereto, over the entire longitudinal extent, i.e., the entire distal-proximal extent of the prosthesis socket. The free space can be in the form of a window completely surrounded by the frame material. In principle, there is also the possibility of, for example, in regions in which the hard, dimensionally stable material of the frame, for example frame pieces formed from prepregs or resin-impregnated glass fiber or carbon fiber precuts, are cut out, with the cutouts and windows then being bridged and, in particular, completely closed by the textile.

The textile can be in the form of a warp-knitted spacer fabric, weft-knitted spacer fabric or woven spacer fabric, which has the advantage that it provides a supporting and padding effect in addition to air permeability. In addition to sufficient strength in the longitudinal extent and transverse extent, a warp-knitted spacer fabric is capable of allowing a padding effect due to the supporting threads and liquid transport due to the air permeability.

In one development of the invention, the textile is adhesively bonded and/or thermally bonded to the frame. The textile can, for example, be adhered to the frame prior to curing of the prepregs and held in place by the resin portion of the prepregs. Thereafter, the prosthesis socket with the adhered textile components or the adhered textile component is then heated under application of a vacuum to an individual model of the stump or to a ready-made model, so that the frame elements cure and the textile is joined directly to the material of the frame. As a result, work steps are saved and a washable prosthesis socket having sufficient strength for fixation to a stump and for accommodation of further prosthesis components is provided.

In one development of the invention, the frame is formed from prepregs which are applied on the outer side of the textile that faces away from the user. As a result, the sharp-edged edges of the frame, which can occur during the production and processing of fiber-reinforced composites, cannot come into direct contact with the user or with a liner worn underneath, thereby reducing the risk of injury or avoiding damage to the liner. Furthermore, the textile additionally provides ventilation and padding on the inner side.

In one development of the invention, at least one material is arranged and incorporated as a further ply, in particular an intermediate ply between the fiber-reinforced plastic, in particular prepreg, and the textile, wherein, when the prosthesis socket is finished, the material or materials has/have a degree of hardness between the degree of hardness of the fiber-reinforced plastic or prepreg and the degree of hardness of the textile. In addition or as an alternative, the material can be attached as a surface ply on the outer side to the fiber-reinforced plastic and/or on the inner side to the textile and/or to an edge of the prosthesis socket as padding in order to cover and pad the sometimes sharp edges of the cured prepregs. The material or materials as an intermediate material or surface material or the padding material or padding materials allow an improved connection to, firstly, the fiber-reinforced plastic or the prepregs and, secondly, the textile, and also a smoother transition between the comparatively soft textile and the very hard frame socket or prepregs in the finished state. The additional material as a surface ply and/or padding serves to protect both the prosthesis component and the user, i.e., as a protective cover, and also as a means of increasing comfort that covers the hard and sometimes sharp-edged edges. The additional material or the intermediate, surface or padding materials can be, for example, one or more elastomers with or without a modified surface, thereby making it possible to make the frame edges softer and to cut out places for fitting electrodes. The elastomer can, for example, be an initially uncrosslinked or slightly crosslinked silicone or an initially uncrosslinked or slightly crosslinked rubber or comprise an initially uncrosslinked or slightly crosslinked rubber or an initially uncrosslinked or slightly crosslinked silicone that is applied to the textile together with the prepregs, is fixed on the textile by means of the resin of the prepreg, and crosslinks and cures under the action of heat during finishing of the prosthesis socket. The intermediate ply remains elastic to a certain extent and its hardness is between that of the textile and that of the cured frame parts. The material of the intermediate ply can also be arranged as a padding material on the side of the frame or a frame edge that is facing away from the textile and can serve as padding or as a base for fixing further components, for example electrodes.

The frame can be joined to the textile—and, in the case of arrangement of at least one intermediate ply, surface ply and/or padding, also the intermediate ply, the surface ply and/or the padding and the textile—via resin from prepregs forming the frame. Further adhesives are are joined for attachment of the textile, with or without interposition of additional materials composed of elastomers such as rubber or the like.

Preferably, when the prosthesis socket has been assembled and is finished, the resin from the prepregs or the other fiber-reinforced base materials does not penetrate as far as the inner side of the textile, i.e., not as far as the surface which is in contact with the user or which is on the side facing the user. As a result, the cured resin is prevented from causing skin irritation, impairing wearing comfort and also impeding air permeability.

In one development of the invention, the textile is in the form of a sleeve or a distally closed liner which, for example, is first pulled onto a model or an impression of the stump and forms the basis for laying the prepregs on the outer side. The sleeve and/or the liner having the distally closed end can be in the form of prefabricated mass-produced articles and pulled onto the model or the cast of the stump.

In the method according to the invention for producing a prosthesis socket, a textile is applied to a stump model, at least one prepreg is applied on the outer side of the textile to form a frame having at least one free space such that the textile at least partially bridges the free space, wherein the textile and the at least one prepreg are then thermally bonded at an elevated temperature and under application of a vacuum to form a prosthesis socket. The method makes it possible to directly join the prepregs or the at least one prepreg and the textile material, with or without arrangement of at least one intermediate ply, for example a film or a fiber composite material having an elastomer component, since the resin from the prepregs joins to the textile directly or joins to the textile or at least a portion of the textile via the intermediate ply.

Advantageously, the textile used is a warp-knitted spacer fabric, weft-knitted spacer fabric or woven spacer fabric which comprises an upper side of the fabric and a lower side of the fabric having supporting threads arranged in between. The resin from the prepregs can then be joined to the upper or lower side of the fabric and, after curing, establish the connection between the subsequently cured material and the textile.

According to one way of producing the prosthesis socket, the textile is pulled onto the stump on the patient and contact areas for the at least one prepreg or the at least one prepreg precut are marked before the prepregs are applied or placed on the outer side of the textile. As a result, it is ensured that, after the textile has been pulled onto the stump model molded from the actual stump, the precuts composed of the resin-impregnated fiber materials are positioned at the correct places.

Preferably, the textile is pulled onto the stump model such that the stump model is at least wrapped up, advantageously completely wrapped up, in the textile, and so an at least circumferentially closed textile can be placed around the stump or stump model.

The at least one free space formed by the frame is preferably arranged medially or laterally. In one embodiment, at least one closed, distal cap is formed on the prosthesis socket via the prepreg or the fiber-reinforced plastic plies in order to provide sufficient stability in the connection region for the further prosthesis component, for example a joint or a receptacle for, for example, a prosthetic hand. Moreover, the closed, distal cap allows a particularly rigid and stable configuration of the distal region of the prosthesis socket that is bordered by optionally elastic regions of the prosthesis socket in the form of the frames or frame sections, between which the textile is arranged in order to provide a closed-walled receptacle for the stump.

Prior to thermal bonding, at least one further ply, for example intermediate ply or surface ply, is placed on and/or between the textile and the prepregs and/or a padding material is placed around the protheses socket edge and they are cured together in order to create space for cables and/or electrodes or the like, to provide protection and also to provide padding. The further ply of material and/or the padding can contain or consist of at least one initially uncrosslinked or slightly crosslinked elastomer and be provided with or without a coating which allows or facilitates adherence to the prepreg or the fiber composite material and/or the textile. After thermal bonding, the textile, the intermediate layer and/or padding layer and the fiber composite material are permanently joined to one another.

### BRIEF DESCRIPTION OF THE DRAWINGS

Exemplary embodiments of the invention will be more particularly elucidated below with reference to the appended figures, in which:

FIG. 1—shows a schematic depiction of a prosthesis socket in a side view;

FIG. 2—shows a rear partial view of the prosthesis socket according to FIG. 1;

FIG. 3—shows a schematic sectional view through a region of the prosthesis socket; and

FIG. 4—shows a variant of FIG. 3.

### DETAILED DESCRIPTION

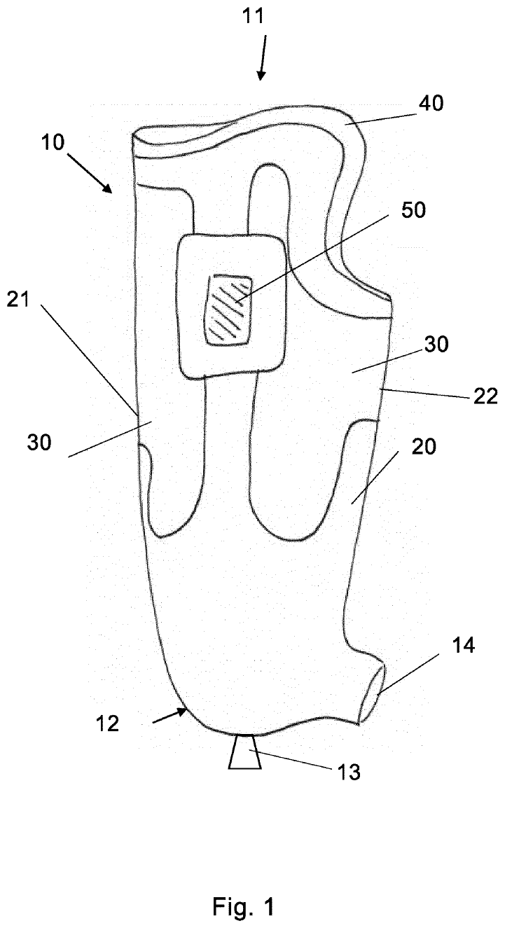

FIG. 1 shows a schematic depiction of a prosthesis socket 10 in a side view. In the exemplary embodiment depicted, the prosthesis socket 10 is in the form of a forearm socket which is to be placed on a forearm stump. To this end, the prosthesis socket 10 is provided with a proximal entry opening 11 in which the forearm stump can be inserted. The prosthesis socket 10 comprises a distal, closed end 12 on which an attachment device 13 for a prosthetic hand is arranged. The attachment device 13 is a schematic depiction of a device for attachment of an adapter to which the prosthetic hand or a prosthetic wrist can be attached. In one embodiment of the prosthesis socket 10, for example in the form of a thigh socket, a prosthetic knee joint is advantageously and usually attached via a pyramid adapter with a corresponding attachment device on the prosthetic knee joint. The pyramid adapter is then usually formed in the region of the distal end of the closed prosthesis socket 10. In the case of an embodiment in the form of a lower leg socket, other attachment devices can also be provided for fixing a prosthetic foot. Since the attachment means for prosthetic hands should take up as little space as possible, a prosthetic wrist is advantageously and usually attached via a lamination ring. However, an attachment device other than a lamination ring can also be provided for fixing the prosthetic hand to the forearm socket. The attachment device 13 depicted can also be oriented differently on the prosthesis socket 10.

The prosthesis socket 10 is in the form of a frame socket and comprises a frame 20 composed of a fiber-reinforced plastic. The frame 20 is made up of so-called prepregs, which are cut to the desired shape and are placed on a stump model, which is not depicted. The prepregs are resin-impregnated fiber wovens which adhere to one another. Because of the excess resin on the prepregs, the precuts also adhere on whatever is the substrate. In the exemplary embodiment depicted, the frame socket comprises two free spaces 21, 22 in the form of windows. A first window 21 is formed in the rear or posterior region, and the second window or the second free space 22 is formed in the frontal or anterior region. The distal end region 12 is closed and encompasses the blunt end around its entire circumference. Formed in the frame 20 proximally to the closed end 12 is a donning hole 14, through which a donning aid pulled over a liner, for example a sleeve composed of a nonadherent material, can be removed after insertion into the prostheses socket 10. The two free spaces 21, 22 are completely bridged and closed by a textile 30. In the exemplary embodiment depicted, the textile 30 is in the form of a warp-knitted spacer fabric arranged on the inner side of the frame 20. The textile 30 is first attached to the prepregs by means of the excess resin present on the surface of the as yet uncured prepregs and is then, during finishing of the prosthesis socket 10 under application of a negative pressure or vacuum and at an elevated temperature, permanently joined to the fiber composite material that is curing. When producing the prosthesis socket 10, the vacuum is preferably set such that the resin does not penetrate as far as the inner side of the textile 30, but that a soft inner surface of the textile continues to be ensured. The vacuum or the negative pressure makes it possible to adjust the contact force of the prepregs on the textile and to thus alter the degree of penetration by the resin.

The prosthesis socket 10 is produced, for example, by taking an impression of the stump of the limb and creating a positive model of the stump from said impression. The positive model can be made of plaster, for example. A standard liner or a custom-made liner is pulled onto the plaster model, such that it rests against the surface of the plaster model as smoothly as possible. The prepregs are placed onto the outer surface of the liner and fixed thereto via the bonding power of the excess resin such that the frame 20 is formed. The free spaces 21, 22 or frame windows can be marked on the textile 30 or the liner beforehand. Those regions on which no prepregs are to be placed and which are not to be covered by the fiber composite material can be marked either on the plaster model or directly on the limb stump. To this end, the liner or the textile 30 is pulled onto the limb stump. As a result, it is possible to take into account individual wishes of users and, for example, to identify particularly sensitive regions directly on the subsequent user of the prosthesis socket. One or more plies of an additional material, in the form of an intermediate material 40 in this embodiment, can be arranged between the prepregs or the plies of prepregs and the textile 30. Preferably, the intermediate material 40 is an elastomer or comprises at least one elastomer component. The intermediate material 40 advantageously joins to the material of the prepregs and also to the textile 30. There is also the option of choosing the intermediate material 40 such that the resin of the prepregs penetrates through the intermediate ply, for example because of the presence of perforation or because the material is permeable to the resin. The intermediate material 40 provides padding and is preferably elastic. Furthermore, the intermediate material 40 can be used to accommodate lines and/or electrodes, and so, for example, electrodes arranged inside the prosthesis socket 10 for recording myoelectric signals can be easily integrated.

Electrode windows 50 can be formed on the frame 20 on the outer side of the prosthesis socket 10. In the exemplary embodiment depicted, only one electrode window 50, which serves to accommodate electrodes or pairs of electrodes, is depicted. For example, control signals from the limb are received via the electrodes, processed in a control unit comprising processor and memory, and used to activate or deactivate drives.

After completion of the frame contour via the prepregs on the outer side of the textile 30, a vacuum is applied. To this end, the model with the textile 30, with or without the intermediate ply or the intermediate plies, and the as yet uncured fiber composite materials is, for example, wrapped in a plastic cover on the model. The plastic cover is evacuated and the model is heated. The vacuum and the heat are maintained until the prepregs have cured to yield a dimensionally stable frame 20.

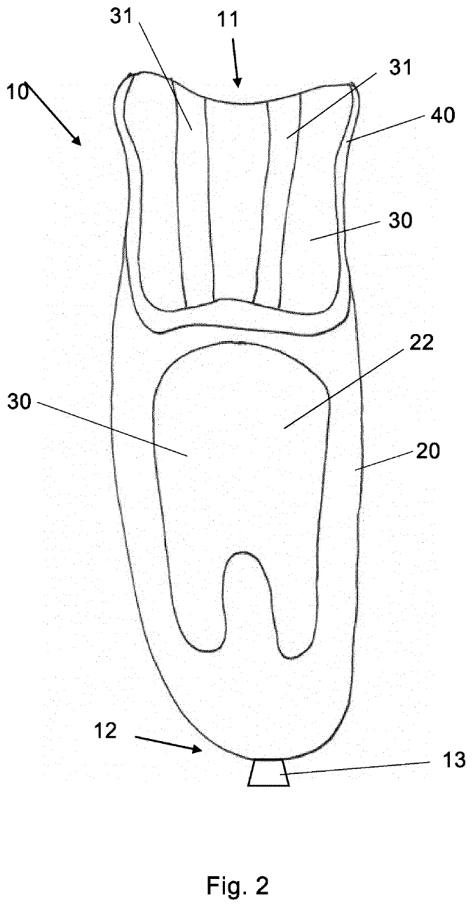

FIG. 2 shows a detailed rear view of the prosthesis socket 10. It can be seen that the textile 30 is arranged and attached on the inner side of the frame 20. A coating 31 can be applied on the inner side of the textile 30 to improve the adherence of the textile 30 to the limb, for example the forearm stump. The coating 31 in the exemplary embodiment depicted consists of silicone and runs in two strips extending from the proximal entry opening 11 up to the distal end region, which is not depicted. The resin from the prepregs forming the dimensionally stable frame 20 has not penetrated as far as the inner surface of the textile 30. The textile 30 is directly joined to the prepregs via the resin from the prepregs. The free space 22 on the back of the prosthesis socket 10 is completely covered by the textile 30, and so the prosthesis socket 11 completely encloses the stump when the prosthesis socket 10 is put on.

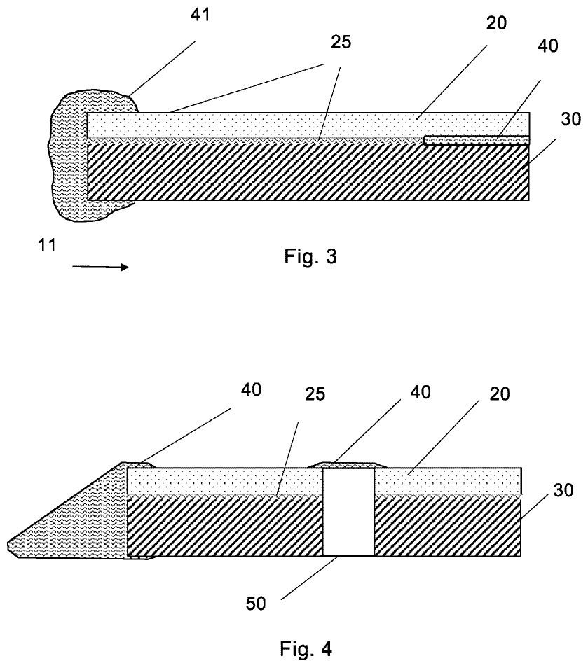

FIG. 3 depicts the connection of a textile 30 to the frame 20 in a schematic sectional view. Moreover, a further material or a further material ply is arranged on the frame 20 as intermediate material 40, this being in the exemplary embodiment depicted, at the left end, on the side of the frame 20 facing away from the textile 30 and, at the right end, between the frame 20 and the textile 30.

The frame 20 composed of a fiber-reinforced plastic, for example composed of prepregs, has, in its processable state, a tacky surface, which, as described above, is caused by the excess resin. The tack will disappear in a subsequent processing step by curing and crosslinking at elevated temperatures under vacuum. The textile 30 is placed on a surface of the frame 20 and is permanently and directly fixed to the frame 20 by the resin in a transition region 25 upon curing or thermal bonding. In the same way, the intermediate material 40 is fixed to the frame 20 on the side of the frame 20 facing away from the textile 30; here too, the fixing is achieved via the resin on the prepregs and the curing or thermal bonding during further processing. The intermediate material 40 can also be arranged between the textile 30 and the frame 20, as shown at the right end of FIG. 3. The resin then completely penetrates the intermediate material 40 and also joins to a portion of the textile 30, for example to the side of the fabric facing the frame 20. The textile 30 is thus fixed to the frame 20 via the intermediate material 40. The resin preferably does not completely penetrate the textile 30 in order to avoid the presence of cured resin on the inner surface of the prosthesis socket 10.

Furthermore, in FIG. 3, padding material 41 is arranged at the edge of the prosthesis socket, which padding material was thermally bonded together with the prepregs. The padding material can be the same material as the material of the intermediate ply 40 and can consist of or comprise an elastomer which is cured in its final state.

FIG. 4 shows a further exemplary embodiment of the invention. FIG. 4 is a variant of the embodiment according to FIG. 3 and likewise shows a schematic sectional view through a proximal region of a prosthesis socket. A textile 30 is positioned on the inner side of the prosthesis socket and is joined to the dimensionally stable frame 20 of the prosthesis socket via a resin layer in the transition region 25. The resin layer penetrates both the textile 30 and the frame 20 in the transition region 25 because the resin is provided from the material of the frame 20 during the production of the prosthesis socket. Excess resin from the starting materials penetrates into the textile 30 at least partially and brings about a permanent and secure connection of the textile 30 to the cured frame 20. Arranged inside the prosthesis socket is a window 50 or a cut-out, into which, for example, an electrode, a sensor, an actuator or the like can be inserted. On the outer side of the frame 20, i.e., on the side facing away from the textile 30, a further material 40 is arranged around the opening or the window 50, which material in its final state consists of a cured elastomer or contains a cured elastomer. This further material ply is applied on the outer side of the frame 40 as a surface ply 40 and forms the uppermost layer, i.e., the layer furthest away from the skin. The surface ply 40 can be applied before or after the introduction of the window 50, and the purpose thereof is that the window 50 can be introduced more easily in order to position electrodes or other devices in the prosthesis socket; furthermore, there are advantages in covering the edge of the hole and in fixing the electrode or the further component within the opening. The surface ply 40 can also be closed or have an opening smaller than the window 50 or surround the edge of the opening.

At the proximal end or in the region of the prosthesis opening 11, the further material 40 is in the form of a combination of padding and surface ply 40. Both on the inner side of the textile 30, as surface facing the skin, and on the outer side of the frame 20, a layer or a region of the additional material composed of cured elastomer or of material containing cured elastomer is applied in order to protect the edge region of the respective frame 20 and of the textile 30. Furthermore, what is formed on the end face is a lip or padding which protects against the hard edge of the frame 20 during insertion of the limb. Therefore, this edge coating on the outer side, the end face and the inner side means that, firstly, the patient and a more comfortable feel during wearing is achieved and, secondly, the prosthesis socket is protected from mechanical damage.

1. A prosthesis socket comprising a proximal entry opening, a distal end, and an attachment device arranged on the distal end for a prosthesis component, the prosthesis socket further comprising a frame composed of a fiber-reinforced plastic having at least one free space and a textile layer bridging the free space and fixed to the frame, and still further comprising a further ply of a material having a hardness between that of the frame and that of the textile layer arranged between the frame and the textile layer as an intermediate ply, as a surface ply and/or at a prosthesis socket edge as padding.

2. The prosthesis socket as claimed in claim 1, wherein the textile layer completely covers the free space.

3. The prosthesis socket as claimed in claim 1, wherein the textile layer is in the form of a warp-knitted spacer fabric, weft-knitted spacer fabric or woven spacer fabric.

4. The prosthesis socket as claimed in claim 1, wherein the textile layer is adhesively bonded and/or thermally bonded to the frame.

5. The prosthesis socket as claimed in claim 1, wherein the frame is formed from prepregs which are applied on an outer side of the textile layer that faces away from a user.

6. The prosthesis socket as claimed in claim 1, wherein the further ply is arranged on the textile layer and/or on the frame.

7. The prosthesis socket as claimed in claim 1, wherein the material of the further ply or of the padding consists of cured elastomer or contains cured elastomer.

8. The prosthesis socket as claimed in claim 1, wherein the frame is joined to the textile layer and, if applicable, to the further ply via resin from prepregs forming the frame.

9. The prosthesis socket as claimed in claim 8, wherein in the assembled state, resin does not penetrate the textile layer to the inner side.

10. The prosthesis socket as claimed in claim 1, wherein the textile layer is in the form of a sleeve or a distally closed liner.

11. A method for producing a prosthesis socket, in which a textile layer is applied to a stump model, at least one prepreg is applied on the outer side of the textile layer to form a frame having at least one free space such that the textile layer at least partially bridges the free space, and the textile layer and the at least one prepreg are then thermally bonded at an elevated temperature and under application of a vacuum to form a prosthesis socket, wherein prior to thermal bonding, at least one further ply is placed between the textile layer and the prepregs as an intermediate layer, on the prepregs and/or the textile layer as a surface layer and/or padding is placed around a prosthesis socket edge and they are cured together.

12. The method as claimed in claim 11, wherein the textile layer used is a warp-knitted spacer fabric, weft-knitted spacer fabric or woven spacer fabric.

13. The method as claimed in claim 11, wherein before being applied to the stump model, the textile layer is pulled onto a stump and contact areas for the at least one prepreg are marked.

14. The method as claimed in claim 11, wherein the stump model is wrapped up in the textile layer.

15. The method as claimed in claim 11, wherein the at least one free space is arranged medially or laterally.

16. The method as claimed in claim 11, wherein a closed, distal cap is formed on the prosthesis socket via at least one prepreg.

17. The method as claimed in claim 11, wherein the further ply is arranged on the textile layer and/or on the frame.

18. The method as claimed in claim 11, wherein the further ply and/or the padding contains or consists of at least one uncrosslinked or slightly crosslinked elastomer.

19. A prosthesis socket comprising:
a proximal entry opening;
a distal end; and
an attachment device on the distal end for a prosthesis component; wherein the prosthesis socket includes a frame composed of a fiber-reinforced plastic having at least one free space; and
wherein a textile layer selected from the group consisting of a warp-knitted spacer fabric, a weft-knitted spacer fabric or a woven spacer fabric, is fixed to the frame, completely covering the free space, wherein a material having a hardness between that of the frame and that of the textile layer is arranged as a further ply between the frame and the textile layer as an intermediate ply, as a surface ply and/or at a prosthesis socket edge as padding.

20. The prosthesis socket of claim 19, wherein the textile layer is adhesively bonded and/or thermally bonded to the frame.
